(19) United States
(12) Patent Application Publication
(10) Pub. No.: US 2024/0008786 A1
(43) Pub. Date: Jan. 11, 2024

(54) MULTISENSORY INDEX SYSTEM AND OPERATION METHOD THEREOF

(71) Applicants: Hyundai Motor Company, Seoul (KR); Kia Corporation, Seoul (KR); Industry-University Cooperation Foundation Hanyang University, Seoul (KR)

(72) Inventors: Ki Chang Kim, Suwon (KR); Dong Chul Park, Anyang (KR); Eun Ju Jeong, Seoul (KR); Ji Yeon Shin, Seoul (KR)

(21) Appl. No.: 17/981,405

(22) Filed: Nov. 5, 2022

(30) Foreign Application Priority Data
Jul. 5, 2022 (KR) 10-2022-0082544

(51) Int. Cl. A61B 5/18; G16H 20/70; G06F 16/635; A61M 21/00

(52) U.S. Cl. CPC A61B 5/18; G16H 20/70; G06F 16/636; A61M 21/00; A61M 2021/0027; A61M 2021/0022

(57) ABSTRACT

A multisensory index system and an operation method thereof are provided. The multisensory index system is configured to derive quantitative parameters and qualitative parameters associated with an auditory sense and a tactile sense, and generate a multisensory index by analyzing a correlation between the quantitative parameters and the qualitative parameters associated with the auditory sense and the tactile sense.

### CROSS-REFERENCE TO RELATED APPLICATION(S)

This application claims, under 35 U.S.C. § 119(a), the benefit of Korean Patent Application No. 10-2022-0082544, filed in the Korean Intellectual Property Office on Jul. 5, 2022, the disclosure of which is incorporated herein by reference in its entirety.

### BACKGROUND

### Technical Field

Embodiments of the present disclosure relate to a multisensory index system and an operation method thereof.

### Background

A healthcare system is a technology of identifying a state of a driver, guiding the driver to receive guidance, and alert and perform safe driving in connection with a vehicle system. The healthcare system may collect biometric information such as, for example, electrocardiogram (ECG) data, heart rates, movement of the driver, and the like, using sensors to determine the state of the driver. Furthermore, the healthcare system may recognize a facial expression of the driver using its camera to determine an emotional state of the driver.

### SUMMARY

The present disclosure has been made to solve the above-mentioned problems occurring in the prior art while advantages achieved by the prior art are maintained intact.

An aspect of the present disclosure provides a multisensory index system for providing a multisensory index by analyzing a correlation between auditory stimulation and tactile stimulation and an operation method thereof.

The technical problems to be solved by the present disclosure are not limited to the aforementioned problems, and any other technical problems not mentioned herein will be clearly understood from the following description by those skilled in the art to which the present disclosure pertains.

According to an aspect of the present disclosure, a multisensory index system may comprise a processor that derives quantitative parameters and qualitative parameters associated with an auditory sense and a tactile sense and generates a multisensory index by analyzing a correlation between the quantitative parameters and the qualitative parameters associated with the auditory sense and the tactile sense.

The quantitative parameters associated with the auditory sense and the tactile sense may comprise a zero crossing rate (ZCR) and a Mel-frequency cepstral coefficient (MFCC).

The ZCR may be used as an auditory and tactile recognition function.

The MFCC may be used for speaker verification and music genre classification.

The qualitative parameters associated with the auditory sense may comprise loudness, timbre, and/or pitch.

The qualitative parameters associated with the tactile sense may comprise intensity, acuity, and/or a location.

The multisensory index may be divided into five stages.

The processor may be configured to determine a first stage when the result of analyzing the correlation is 1.1 to 2.0, may be configured to determine a second stage when the result of analyzing the correlation is 2.1 to 3.0, may be configured to determine a third stage when the result of analyzing the correlation is 3.1 to 4.0, may be configured to determine a fourth stage when the result of analyzing the correlation is 4.1 to 5.0, and may be configured to determine a fifth stage when the result of analyzing the correlation is 5.1 to 6.0.

The processor may be configured to provide an emotional care solution based on the multisensory index.

According to another aspect of the present disclosure, an operation method of a multisensory index system may comprise deriving quantitative parameters and qualitative parameters associated with an auditory sense and a tactile sense and generating a multisensory index by analyzing a correlation between the quantitative parameters and the qualitative parameters associated with the auditory sense and the tactile sense.

The quantitative parameters associated with the auditory sense and the tactile sense may comprise a zero crossing rate (ZCR) and a Mel-frequency cepstral coefficient (MFCC).

The ZCR may be used as an auditory and tactile recognition function.

The MFCC may be used for speaker verification and music genre classification.

The qualitative parameters associated with the auditory sense may comprise loudness, timbre, and/or pitch.

The qualitative parameters associated with the tactile sense may comprise intensity, acuity, and/or a location.

The multisensory index may be divided into five stages.

The generating of the multisensory index may comprise determining a first stage when the result of analyzing the correlation is 1.1 to 2.0, determining a second stage when the result of analyzing the correlation is 2.1 to 3.0, determining a third stage when the result of analyzing the correlation is 3.1 to 4.0, determining a fourth stage when the result of analyzing the correlation is 4.1 to 5.0, and determining a fifth stage when the result of analyzing the correlation is 5.1 to 6.0.

The operation method may further comprise providing an emotional care solution based on the multisensory index.

### BRIEF DESCRIPTION OF THE DRAWINGS

The above and other objects, features and advantages of the present disclosure will be more apparent from the following detailed description taken in conjunction with the accompanying drawings:

FIG. 1 is a block diagram illustrating a configuration of a multisensory index system according to exemplary embodiments of the present disclosure;

FIG. 2 is a flowchart illustrating an emotional care solution method based on a multisensory index according to exemplary embodiments of the present disclosure;

FIG. 3 is a drawing for describing a process of deriving quantitative and qualitative parameters associated with an auditory sense and a tactile sense according to exemplary embodiments of the present disclosure;

FIG. 4 is a drawing for describing a process of generating a multisensory index according to exemplary embodiments of the present disclosure; and

FIG. 5 is a flowchart illustrating an emotional care providing method according to exemplary embodiments of the present disclosure.

### DETAILED DESCRIPTION

Hereinafter, some embodiments of the present disclosure will be described in detail with reference to the exemplary drawings. In the drawings, the same reference numerals will be used throughout to designate the same or equivalent elements. In addition, a detailed description of well-known features or functions will be ruled out in order not to unnecessarily obscure the gist of the present disclosure.

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g. fuels derived from resources other than petroleum). As referred to herein, a hybrid vehicle is a vehicle that has two or more sources of power, for example both gasoline-powered and electric-powered vehicles.

The terminology used herein is for the purpose of describing particular embodiments only and is not intended to be limiting of the disclosure. As used herein, the singular forms “a,” “an” and “the” are intended to include the plural forms as well, unless the context clearly indicates otherwise. These terms are merely intended to distinguish one component from another component, and the terms do not limit the nature, sequence or order of the constituent components. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Throughout the specification, unless explicitly described to the contrary, the word “comprise” and variations such as “comprises” or “comprising” will be understood to imply the inclusion of stated elements but not the exclusion of any other elements. In addition, the terms “unit”, “-er”, “-or”, and “module” described in the specification mean units for processing at least one function and operation, and can be implemented by hardware components or software components and combinations thereof.

Although exemplary embodiment is described as using a plurality of units to perform the exemplary process, it is understood that the exemplary processes may also be performed by one or plurality of modules. Additionally, it is understood that the term controller/control unit refers to a hardware device that includes a memory and a processor and is specifically programmed to execute the processes described herein. The memory is configured to store the modules and the processor is specifically configured to execute said modules to perform one or more processes which are described further below.

Further, the control logic of the present disclosure may be embodied as non-transitory computer readable media on a computer readable medium containing executable program instructions executed by a processor, controller or the like. Examples of computer readable media include, but are not limited to, ROM, RAM, compact disc (CD)-ROMs, magnetic tapes, floppy disks, flash drives, smart cards and optical data storage devices. The computer readable medium can also be distributed in network coupled computer systems so that the computer readable media is stored and executed in a distributed fashion, e.g., by a telematics server or a Controller Area Network (CAN).

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about”. In describing the components of the embodiment according to the present disclosure, terms such as first, second, “A”, “B”, (a), (b), and the like may be used. These terms are only used to distinguish one element from another element, but do not limit the corresponding elements irrespective of the order or priority of the corresponding elements. Furthermore, unless otherwise defined, all terms including technical and scientific terms used herein are to be interpreted as is customary in the art to which the present disclosure belongs. Such terms as those defined in a generally used dictionary are to be interpreted as having meanings equal to the contextual meanings in the relevant field of art, and are not to be interpreted as having ideal or excessively formal meanings unless clearly defined as having such in the present application.

The multisensory in the specification refers to a technology for developing a system with regard to an auditory sense, a visual sense, and a tactile sense in terms of emotional quality. The specification proposes a process of a multisensory index for implementing a mood curator function in a vehicle environment. The mood curator function helps vehicle passengers refresh themselves depending on their current emotional states. In other words, the mood curator function may be configured to stimulate the five senses of a passenger in a combination of systems (e.g., music, a fragrance, lighting, a massage, a monitor screen, and a curtain) in the vehicle to improve an emotional state of the passenger.

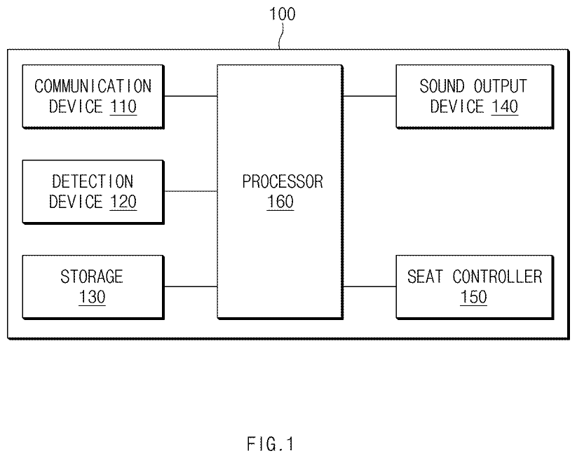

FIG. 1 is a block diagram illustrating a configuration of a multisensory index system according to exemplary embodiments of the present disclosure.

Referring to FIG. 1, a multisensory index system (hereinafter referred to as a “system”) 100 may comprise a communication device 110, a detection device 120, storage 130, a sound output device 140, a seat controller 150, and/or a processor 160.

The communication device 110 may be configured to assist the system 100 to perform wired communication and/or a wireless communication with an electronic device (e.g., a smartphone, an electronic control unit (ECU), a tablet, a personal computer, or the like) which is located inside and/or outside a vehicle. The communication device 110 may comprise a transceiver which transmits and/or receives a signal (or data) using at least one antenna.

The detection device 120 may be configured to detect vehicle information (e.g., driving information and/or vehicle interior and exterior environment information), driver information, passenger information, and/or the like. The detection device 120 may be configured to detect vehicle information, such as a vehicle speed, seat information, a motor revolution per minute (RPM), an accelerator pedal opening amount, a throttle opening amount, a vehicle interior temperature, and/or a vehicle exterior temperature, using at least one sensor and/or at least one ECU, which are/is mounted on the vehicle. An accelerator position sensor (APS), a throttle position sensor, a global positioning system (GPS) sensor, a wheel speed sensor, a temperature sensor, a microphone, an image sensor, an advanced driver assistance system (ADAS) sensor, a 3-axis accelerometer, an inertial measurement unit (IMU), and/or the like may be used as the at least one sensor. The at least one ECU may be a motor control unit (MCU), a vehicle control unit (VCU), and/or the like. The detection device 120 may be configured to detect driver information and passenger information using a pressure sensor, an ultrasonic sensor, a radar, an image sensor, a microphone, a driver monitoring system (DMS), and/or the like. The detection device 120 may be configured to detect a biometric signal (e.g., an electroencephalogram (EEG), a heart rate, a respiratory rate, and/or the like) using a contact sensor or a non-contact sensor.

The storage 130 may be configured to store a sound (or a sound source) such as a music sound (or music content), a virtual sound, and/or a driving sound. The storage 130 may be configured to store an emotional model, a multisensory index, and/or the like. The storage 130 may be a non-transitory storage medium which stores instructions executed by the processor 160. The storage 130 may comprise at least one of storage media such as a random access memory (RAM), a static RAM (SRAM), a read only memory (ROM), a programmable ROM (PROM), an electrically erasable and programmable ROM (EEPROM), an erasable and programmable ROM (EPROM), a hard disk drive (HDD), a solid state disk (SSD), an embedded multimedia card (eMMC), universal flash storage (UFS), or web storage.

The sound output device 140 may be configured to play and output a sound source which is previously stored or is streamed in real time to the outside. The sound output device 140 may comprise an amplifier, speakers (e.g., a twitter, a woofer, a subwoofer, and the like), and/or the like. The amplifier may be configured to amplify an electrical signal of a sound played from the sound output device 140. A plurality of speakers may be installed at different positions inside and/or outside the vehicle. The speaker may be configured to convert the electrical signal amplified by the amplifier into a sound wave.

The seat controller 150 may be configured to control at least one vibrator mounted on a vehicle seat to generate a vibration (or a vibration signal). The seat controller 150 may be configured to adjust a vibration pattern, vibration intensity, a vibration frequency, and/or the like. At least one vibrator may be installed at a specific position of the vehicle seat, for example, a seat back, a seat cushion, a leg rest, and/or the like.

The seat controller 150 may be configured to control a vibrator, an actuator, and/or the like in a neck pillow to provide a haptic effect to a neck of a passenger (or a user) who sits on a vehicle seat. The neck pillow may be removably made at a boundary between a seat back and a headrest of the vehicle seat.

The processor 160 may be electrically connected with the respective components 110 to 150. The processor 160 may be configured to control operations of the respective components 110 to 150. The processor 160 may comprise at least one of processing devices such as an application specific integrated circuit (ASIC), a digital signal processor (DSP), programmable logic devices (PLD), field programmable gate arrays (FPGAs), a central processing unit (CPU), microcontrollers, or microprocessors.

The processor 160 may be configured to control the sound output device 140 depending on an input of the user (e.g., a driver, a passenger, or the like) in the interior of the vehicle to play music content. The user may input data through a user interface (e.g., a touch pad, a keypad, a touch screen, or the like). The processor 160 may be configured to determine an emotional care solution matched with the played music content based on the multisensory index. The emotional care solution may be configured to determine a tactile stimulation signal pattern (i.e., a magnitude, a period, and the like of the signal) such as a vibration and/or haptics of the vehicle seat and the neck pillow.

In other words, the processor 160 may be configured to provide tactile (or vibrational and/or haptic) emotion care based on a sound, for example, music content, a virtual sound, or the like, which is played in the vehicle. The processor 160 may be configured to implement vibration and haptics based on the sound in a seat, a neck pillow, and the like of the vehicle.

The processor 160 may be configured to derive quantitative parameters and qualitative parameters for an auditory sense and a tactile sense to analyze an emotional evaluation correlation factor. The processor 160 may be configured to select quantitative parameters for an auditory sense and a tactile sense by analyzing a musical variable. The processor 160 may be configured to extract a zero crossing rate (ZCR) and a Mel-frequency cepstral coefficient (MFCC), which are factors with the highest correlation by analyzing a correlation between an auditory parameter and a tactile parameter among the selected parameters.

The processor 160 may be configured to derive a correlation equation between qualitative parameters for an auditory sense (or auditory qualitative parameters) and qualitative parameters for a tactile sense (or tactile qualitative parameters) by means of a statistical analysis. At this time, the auditory qualitative parameters and the tactile qualitative parameters may be representative factors capable of matching an auditory sense with a tactile sense, which are selected among a plurality of parameters. The auditory qualitative parameters may comprise loudness, timbre, pitch, and the like. The timbre may be one of the three elements of sound, which may represent characteristics in which the sound feels different depending on a sounding body. The tactile qualitative parameters may comprise intensity, acuity, a location, and the like. Herein, the acuity may be tactile perception sensitivity, which may be used to increase the speed of nerve cell transmission to increase efficiency.

The processor 160 may be configured to establish three concept-based service solutions, that is, three types of emotion modeling based on driver's emotion using a pleasure arousal dominance (PAD) model. The PAD model may be configured to represent an emotional state using pleasure, arousal, and dominance. Herein, the dominance may be associated with harmonics. The three types of emotion modeling based on the driver's emotion may comprise a stress relief (or safe driving) solution, a meditation (or healthy driving) solution, and a healing (or fun driving) solution. The stress relief solution may provide music stimulation for two to three minutes, which may assign safe feeling of driving at the same time as performing a comfortable interaction with a passenger The meditation solution may provide music stimulation for two to third minutes, which may provide a comfortable rest, and vibration pattern stimulation synchronized with music which is able to stimulates the user to immerse himself or herself in a specific state (e.g. sleep). The healing solution may provide music stimulation for two to third minutes in an exciting feeling and continuous vibration pattern stimulation for stimulating hedonistic sensual pleasure. The processor 160 may be configured to provide an emotional care solution based on driver emotion modeling, that is, stress relief content, meditation (or rest and relaxation) content, healing (or tension up) content, and the like.

The processor 160 may be configured to generate a multisensory index by analyzing a correlation between the quantitative parameters and the qualitative parameters for the auditory sense and the tactile sense. The processor 160 may be configured to reflect a contribution rate when generating the multisensory index. The multisensory index may be divided into five stages.

When a first stage of the multisensory index based on the driver emotion modeling is determined, the processor 160 may be configured to link to a meditation concept to implement sound-based vibration and haptics in a seat and a neck pillow. When a second stage of the multisensory index based on the driver emotion modeling is determined, the processor 160 may be configured to link to a stress relief concept to implement sound-based vibration and haptics in the seat and the neck pillow. When a third stage of the multisensory index based on the driver emotion modeling is determined, the processor 160 may be configured to implement healing emotion vibration and haptics. When a fourth stage of the multisensory index based on the driver emotion modeling is determined, the processor 160 may be configured to implement a warning signal. When a fifth stage of the multisensory index based on the driver emotion modeling is determined, the processor 160 may be configured to implement personalization or a massage. The processor 160 may be configured to adjust sound-based seat vibration and haptic intensity based on the multisensory index.

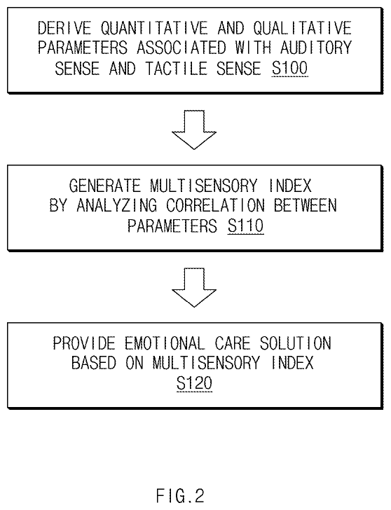

FIG. 2 is a flowchart illustrating an emotional care solution method based on a multisensory index according to exemplary embodiments of the present disclosure;

Referring to FIG. 2, in S100, a processor 160 of FIG. 1 may be configured to derive quantitative and qualitative parameters associated with an auditory sense and a tactile sense. The processor 160 may be configured to select quantitative parameters associated with an auditory sense and a tactile sense by analyzing a musical variable. The processor 160 may be configured to select (or extract) a factor with a high correlation by analyzing a correction between auditory parameters (or auditory sense-related quantitative parameters) and tactile parameters (or tactile sense-related quantitative parameters) among the selected quantitative parameters. The processor 160 may be configured to extract a factor with a high correlation, that is, a zero crossing rate (ZCR) and a Mel-frequency cepstral coefficient (MFCC), among the selected quantitative parameters.

In S110, the processor 160 may be configured to generate a multisensory index by analyzing a correlation between parameters. The processor 160 may be configured to generate the multisensory index based on quantitative parameters of an auditory sense and a tactile sense (or auditory and tactile quantitative parameters), qualitative parameters of an auditory sense (or auditory qualitative parameters), and qualitative parameters of a tactile sense (or tactile qualitative parameters). In other words, the processor 160 may be configured to derive the multisensory index by analyzing a correlation between the quantitative parameters and the qualitative parameters of the auditory sense and the tactile sense. The multisensory index may be divided into five stages based on the result of analyzing the correlation between the quantitative parameters and the qualitative parameters of the auditory sense and the tactile sense.

In S120, the processor 160 may be configured to provide an emotional care solution based on the multisensory index. When the multisensory index is a first stage, the processor 160 may be configured to provide a meditation mode. When the multisensory index is a second stage, the processor 160 may be configured to provide a stress relief mode. When the multisensory index is a third stage, the processor 160 may be configured to provide a healing mode. When the multisensory index is a fourth stage, the processor 160 may be configured to provide a warning signal. When the multisensory index is a fifth stage, the processor 160 may be configured to provide a message. The processor 160 may be configured to control a seat controller 150 of FIG. 1 to adjust intensity of sound-based vibration and haptics corresponding to the emotional care solution.

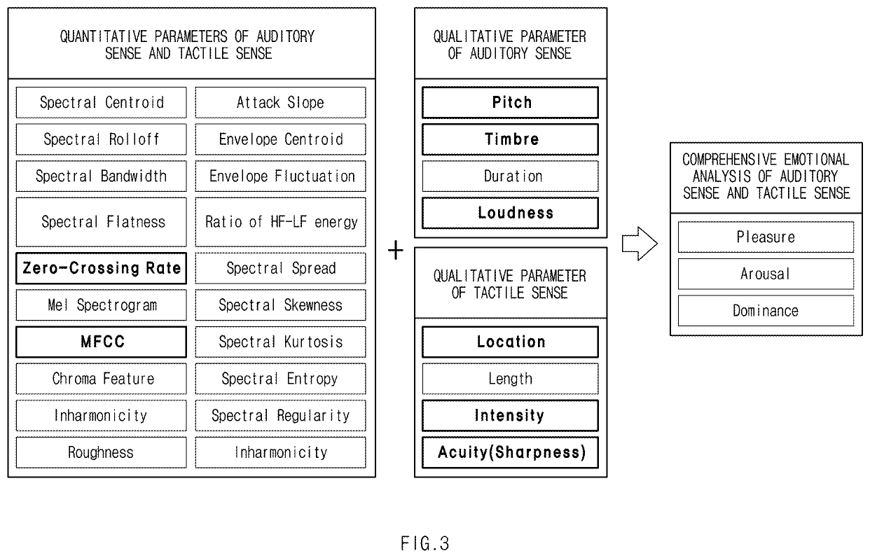

FIG. 3 is a drawing for describing a process of deriving quantitative and qualitative parameters associated with an auditory sense and a tactile sense according to exemplary embodiments of the present disclosure.

Referring to FIG. 3, a processor 160 of FIG. 1 may be configured to select quantitative parameters associated with an auditory sense and a tactile sense by analyzing a musical variable. The processor 160 may be configured to select (or extract) a factor with a high correlation by analyzing a correction between auditory parameters (or auditory sense-related quantitative parameters) and tactile parameters (or tactile sense-related quantitative parameters) among the selected quantitative parameters. The processor 160 may be configured to extract a factor with a high correlation, that is, a zero crossing rate (ZCR) and a Mel-frequency cepstral coefficient (MFCC), among the selected quantitative parameters.

The ZCR refers to a speed at which the signal changes from a positive number to “0” or changes from a negative number to “0”, that is, a rate at which the signal crosses “0”. In the present embodiment, the ZCR is used as an auditory and tactile recognition function. The ZCR may be defined as Equation 1 below.

$$\\ \begin{matrix}ZCR = \frac{1}{T - 1} {\sum }_{t = 1}^{T - 1} ΙΙ \{ {s}_{t} {s}_{t - 1} < 0 \} & Equation 1\end{matrix}$$

Herein, s denotes the input signal and T denotes the length of the signal. II{sₜsₜ₋₁<0} may be used to determine whether the value obtained by multiplying the signal value st of a current sample by the signal value sₜ₋₁ of a previous sample is minus, return “1” when the multiplied value is minus, and return “0” when the multiplied value is not minus.

The present embodiment describes deriving the ZCR as a quantitative parameter, but not limited thereto. The present embodiment may derive a short time zero crossing rate (STZCR) rather than the ZCR as a quantitative parameter. The STZCR Zn may be defined as Equation 2 below.

$$\\ \begin{matrix}{Z}_{n} = {\sum }_{m = - ∞}^{∞} \vert sgn [ x ( m ) ] - sgn [ x ( m - 1 ) ] \vert w ( n - m ) & Equation 2\end{matrix}$$

Herein, x(m) refers to the input signal and w(n-m) refers to the window for energy conversion over time. The STZCR may be used to distinguish voiced speed from unvoiced speed.

The MFCC is a numerical value indicating a unique feature capable of being extracted from an audio signal. The process of extracting the MFCC will be described in brief. First, the audio signal may be divided for each frame (20 ms to 40 ms) to be applied to fast Fourier transform (FFT) and obtain a spectrum (or a frequency component). The FFT is an algorithm for converting a time domain signal into a frequency component. Because of representing sound pressure according to a frequency, the spectrum may be to identify strength and weakness by means of intensity for each frequency band. In other words, a harmonics structure may be inferred from the spectrum. Secondly, a Mel filter bank may be applied to the spectrum to derive a Mel spectrum. The Mel spectrum represents a relationship between a physical frequency and a frequency actually recognized by a person by reflecting a characteristic in which the human auditory organ is more sensitive in the low frequency band than in the high frequency band. Finally, the MFCC may be derived (or extracted) by means of a cepstral analysis in the Mel spectrum. The cepstral analysis is a process of separating a curve connecting formants, that is, a spectral envelope from the spectrum. The formant is a unique feature of a sound, which is a specific frequency band in which the sound resonates. The MFCC may be mainly used for speaker verification, music genre classification, and the like.

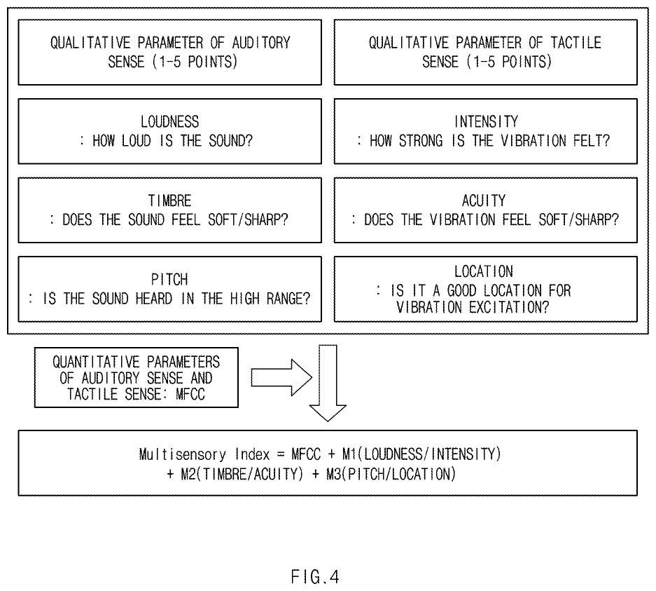

FIG. 4 is a drawing for describing a process of generating a multisensory index according to exemplary embodiments of the present disclosure.

A processor 160 of FIG. 1 may be configured to generate a multisensory index based on quantitative parameters of an auditory sense and a tactile sense (or auditory and tactile quantitative parameters), qualitative parameters of an auditory sense (or auditory qualitative parameters), and qualitative parameters of a tactile sense (or tactile qualitative parameters). In other words, the processor 160 may be configured to derive the multi sensory index by analyzing a correlation between the quantitative parameters and the qualitative parameters of the auditory sense and the tactile sense. The processor 160 may be configured to extract features of the auditory sense and the tactile sense to digitize (e.g., 0.1 to 1.0) the extracted features as a quantitative variable. The processor 160 may be configured to compare an auditory qualitative parameter with a tactile qualitative parameter and may be configured to digitize (e.g., 0 to 1) the compared result. At this time, when the digitized value is greater than “1”, the processor 160 may be configured to limit (or filter) an upper limit to “1”. Furthermore, the processor 160 may be configured to reflect contribution rates M1, M2, and M3 for a qualitative parameter. Herein, the contribution rates M1, M2, and M3 are derived by means of a regression analysis and a statistical analysis for user emotion evaluation. In detail, a variable is selected by means of the regression analysis to derive a correlation equation by means of model diagnosis. A user emotion evaluation database may be statistically analyzed as Table 1 below to derive the contribution rates M1, M2, and M3.

TABLE 1

| | Non-standardized | | | | | | |
|---|---|---|---|---|---|---|---|
| | coefficients | | | | Probabil- | | |
| | | Standard- | Standardized | | ity of | Collinearity | |
| | | ization | coefficients | | signif- | statistics | |
| Factor | B | error | Beta | t | icance | Tolerance | VIF |
| MFCC | 1.0 | 0.02 | | 4.77 | 0.0 | | |
| M1 | 0.625 | 0.02 | 0.406 | 1.25 | 0.0 | 1.0 | 1.0 |
| M2 | 0.195 | 0.02 | 0.127 | 0.39 | 0.0 | 1.0 | 1.0 |
| M3 | 0.180 | 0.02 | 0.117 | 0.36 | 0.0 | 1.0 | 1.0 |

The multisensory index may be divided into five stages based on the result of analyzing the correlation between the quantitative parameters and the qualitative parameters for the auditory sense and the tactile sense. When the result of analyzing the correlation is 1.1 to 2.0, the processor 160 may be configured to determine a first stage. When the result of analyzing the correlation is 2.1 to 3.0, the processor 160 may be configured to determine a second stage. When the result of analyzing the correlation is 3.1 to 4.0, the processor 160 may be configured to determine a third stage. When the result of analyzing the correlation is 4.1 to 5.0, the processor 160 may be configured to determine a fourth stage. When the result of analyzing the correlation is 5.1 to 6.0, the processor 160 may be configured to determine a fifth stage. The five stages of the multisensory index are classified based on a user evaluation database according to actual vehicle feedback based on a driver emotion model.

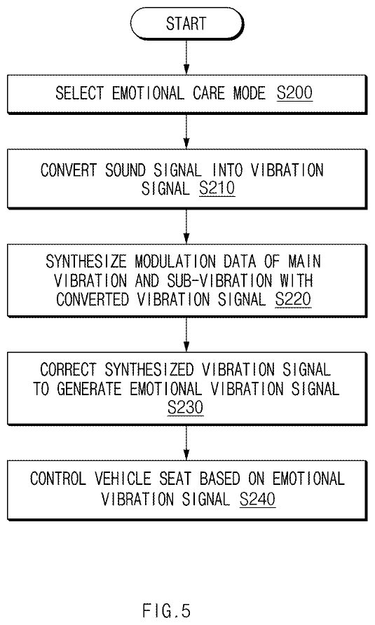

FIG. 5 is a flowchart illustrating an emotional care providing method according to exemplary embodiments of the present disclosure.

In S200, a processor 160 of FIG. 1 may be configured to select an emotional care mode based on at least one of a user input, a driving environment, or a user state. The processor 160 may be configured to determine the emotional care mode depending on a user input. The processor 160 may be configured to determine the emotional care mode based on a vehicle environment and/or an emotional state of a passenger. The processor 160 may be configured to select the emotional care mode based on a pre-training database by an artificial intelligence-based emotional vibration algorithm. The emotional care mode may be divided into a meditation mode, a stress relief mode, and a healing mode.

In S210, the processor 160 may be configured to convert a sound signal into a vibration signal based on the selected emotional care mode. The processor 160 may be configured to implement a vibration multi-mode based on a sound. The vibration multi-mode may comprise a beat machine, a simple beat, a natural beat, a live vocal, and the like.

In S220, the processor 160 may be configured to synthesize modulation data of a main vibration and a sub-vibration with the converted vibration signal. The main vibration may be a sine wave, and the sub-vibration may be a square wave, a triangle wave, and/or a sawtooth wave. The processor 160 may be configured to perform modulation using a modulation scheme of at least one of a pulse amplitude, a pulse width, or a pulse position of the main vibration and the sub-vibration.

In S230, the processor 160 may be configured to correct the synthesized vibration signal to generate an emotional vibration signal. The processor 160 may be configured to determine a frequency value suitable for a back and thighs in the synthesized vibration signal. The processor 160 may be configured to determine a level, a time, or an optimal pattern value of an individual actuator based on the synthesized vibration signal. The processor 160 may be configured to correct a vibration exciting force according to a sitting posture or a driving sound pattern.

In S240, the processor 160 may be configured to control a vehicle seat based on the emotional vibration signal. The processor 160 may be configured to control a seat controller 150 of FIG. 1 to excite a vibration in the vehicle seat.

Embodiments of the present disclosure may be configured to provide a multisensory index by analyzing a correlation between auditory stimulation and tactile stimulation.

Furthermore, embodiments of the present disclosure may be configured to adjust a magnitude and period of a tactile stimulation signal (e.g., vibration, haptics, and/or the like) using multisensory index-based control logic to play a pattern.

Furthermore, embodiments of the present disclosure may be configured to apply a sound-based technology using the five senses to real vehicles without any sense of difference based on the multisensory index.

Hereinabove, although the present disclosure has been described with reference to exemplary embodiments and the accompanying drawings, the present disclosure is not limited thereto, but may be variously modified and altered by those skilled in the art to which the present disclosure pertains without departing from the spirit and scope of the present disclosure claimed in the following claims. Therefore, embodiments of the present disclosure are not intended to limit the technical spirit of the present disclosure, but provided only for the illustrative purpose. The scope of the present disclosure should be construed on the basis of the accompanying claims, and all the technical ideas within the scope equivalent to the claims should be included in the scope of the present disclosure.

What is claimed is:

1. A multisensory index system, comprising:
a processor configured to:
derive quantitative parameters and qualitative parameters associated with an auditory sense and a tactile sense; and
generate a multisensory index by analyzing a correlation between the quantitative parameters and the qualitative parameters associated with the auditory sense and the tactile sense.

2. The multisensory index system of claim 1, wherein the quantitative parameters associated with the auditory sense and the tactile sense comprise a zero crossing rate (ZCR) and a Mel-frequency cepstral coefficient (MFCC).

3. The multisensory index system of claim 2, wherein the ZCR is used as an auditory and tactile recognition function.

4. The multisensory index system of claim 2, wherein the MFCC is used for speaker verification and music genre classification.

5. The multisensory index system of claim 1, wherein the qualitative parameters associated with the auditory sense comprise one or more of the following: loudness; timbre; and pitch.

6. The multisensory index system of claim 1, wherein the qualitative parameters associated with the tactile sense comprise one or more of the following: intensity; acuity; and a location.

7. The multisensory index system of claim 1, wherein the multisensory index is divided into five stages.

8. The multisensory index system of claim 7, wherein the processor is configured to:
determine a first stage when a result of analyzing the correlation is 1.1 to 2.0;
determine a second stage when the result of analyzing the correlation is 2.1 to 3.0;
determine a third stage when the result of analyzing the correlation is 3.1 to 4.0;
determine a fourth stage when the result of analyzing the correlation is 4.1 to 5.0; and
determine a fifth stage when the result of analyzing the correlation is 5.1 to 6.0.

9. The multisensory index system of claim 1, wherein the processor is configured to provide an emotional care solution based on the multisensory index.

10. An operation method of a multisensory index system, the operation method comprising:
deriving, by a processor, quantitative parameters and qualitative parameters associated with an auditory sense and a tactile sense; and
generating, by the processor, a multisensory index by analyzing a correlation between the quantitative parameters and the qualitative parameters associated with the auditory sense and the tactile sense.

11. The operation method of claim 10, wherein the quantitative parameters associated with the auditory sense and the tactile sense comprise a zero crossing rate (ZCR) and a Mel-frequency cepstral coefficient (MFCC).

12. The operation method of claim 11, wherein the ZCR is used as an auditory and tactile recognition function.

13. The operation method of claim 11, wherein the MFCC is used for speaker verification and music genre classification.

14. The operation method of claim 10, wherein the qualitative parameters associated with the auditory sense comprise one or more of the following: loudness; timbre; and pitch.

15. The operation method of claim 10, wherein the qualitative parameters associated with the tactile sense comprise one or more of the following: intensity; acuity; and a location.

16. The operation method of claim 10, wherein the multisensory index is divided into five stages.

17. The operation method of claim 16, wherein the generating of the multisensory index comprises determining, by the processor:
a first stage when a result of analyzing the correlation is 1.1 to 2.0;
a second stage when the result of analyzing the correlation is 2.1 to 3.0;
a third stage when the result of analyzing the correlation is 3.1 to 4.0;
a fourth stage when the result of analyzing the correlation is 4.1 to 5.0; and
a fifth stage when the result of analyzing the correlation is 5.1 to 6.0.

18. The operation method of claim 10, further comprising providing, by the processor, an emotional care solution based on the multisensory index.
